(19) United States
(12) Patent Application Publication
(10) Pub. No.: US 2009/0214583 A1
(43) Pub. Date: Aug. 27, 2009

(54) Epitope-enhancement of a human cd4 hiv epitope

(75) Inventors: Jay A. Berzofsky, Bethesda, MD (US); Takahiro Okazaki, Aobaku (JP)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Government of the United States of America as represented by the Secretary of the Department of

(21) Appl. No.: 11/579,230

(22) PCT Filed: Apr. 27, 2005
(86) PCT No.: PCT/US2005/014569

§ 371 Date: Oct. 30, 2006

Related U.S. Application Data
(60) Provisional application No. 60/567,073, filed on Apr. 30, 2004.

(51) Int. Cl. A61K 39/21; C07K 7/08; C12Q 1/02

(52) U.S. Cl. 424/188.1; 530/326; 424/185.1; 435/29

(57) ABSTRACT

Virus-specific CD4⁺T cell help and CD8⁺ cytotoxic T cell responses are critical for the maintenance of effective immunity in chronic viral infections. The importance of the CD4⁺T cells has been documented in HIV infection. A T1-specific CD4⁺T cell line from a healthy volunteer immunized with a canarypox vector expressing gp120 has been developed. The cell line was restricted to DR13, which is common in the U.S. in both Caucasians and African-Americans and is one of the major haplotypes in Africans. Amino acid substitutions in the T1 epitope were made to induce a stronger epitope-specific CD4⁺T cell response than the original epitope resulting in an improved CD4 epitope. A polypeptide comprising the enhanced CD4 epitope can be used as a component in compositions either alone or in combination with other adjuvants and other immunogenic compositions to induce a more effective immune response to HIV infection.

### CROSS REFERENCE TO RELATED APPLICATIONS

The present application claims benefit of U.S. Provisional Patent Application No. 60/567,073, filed on Apr. 30, 2004, the complete disclosure of which is incorporated herein by reference.

### BACKGROUND OF THE INVENTION

CD4⁺ T cell help plays a critical role in maintaining CTL function in viral infection (Zajac et al., J. Exp. Med., 188:2205 (1998); Walter et al., N. Engl. J. Med., 333:1038 (1995); Cardin et al., J. Exp. Med., 184:863 (1996); Hasenkrug et al., J. Virol., 72:6559 (1998); Rosenberg et al., Science, 278:1447 (1997)). Especially in HIV infection, a quantitative decline in the number of CD4⁺ lymphocytes and a qualitative impairment of their function are observed that lead the patients to acquired immunodeficiency syndrome (AIDS). However, the HIV-specific CD4⁺ T cell response can be recovered after, for example, the highly active anti-retroviral therapy (HAART). This recovery is inversely correlated with HIV viral load (Rosenberg et al., Science, 278:1447 (1997); Rosenberg et al., Nature, 407:523 (2000)).

On the other hand, in mice, CD4⁺ T cells are required for expansion and memory of CD8⁺ CTLs (Janssen et al., Nature, 421:852 (2003); Shedlock et al., Science, 300:337 (2003); Sun et al., Science, 300:339 (2003)), suggesting that stronger CD4⁺ T cell help is required for the maintenance of CTL and control of viremia. Furthermore, these facts suggest the hypothesis that the immune response induced by HIV infection might be insufficient, in part due to the evolution of HIV epitopes under immune selective pressure to evade the immune response. Therefore, the virus might be controlled by a vaccine incorporating improved CD4⁺ epitopes substituted at some amino acids positions to induce a stronger CD4⁺ T cell response for helping HIV-specific CTL proliferation, together with similarly epitope enhanced CTL epitopes. This approach termed “epitope enhancement” could enable the development of a more effective HIV vaccine (Berzofsky, Ann. N.Y. Acad. Sci. USA, 690:256 (1993); Berzofsky et al., Immunol. Rev., 170:151 (1999); Berzofsky et al., Nature Reviews Immunology, 1:209 (2001)). Whereas epitope enhancement has been applied to several peptides binding class I HLA molecules which induce an antigen specific CD8⁺ T cell response, there is little experience doing so for epitopes binding to class II HLA molecules for which binding motifs are less well defined.

The T1 antigen is a 16 amino acid residue peptide (KQIINMWQEVGK AMYA, SEQ ID NO: 1) comprising a CD4 epitope which was the first helper epitope discovered in the HIV envelope protein (Cease et al., Proc. Natl. Acad. Sci. USA, 84:4249 (1987)). Immunization with this epitope can induce an epitope-specific CD4 response in mice and the same epitope is recognized by human T cells from HIV-infected or immunized individuals (Clerici et al., Nature, 339:383 (1989); Berzofsky et al., Nature, 334:706 (1988)). The T1 epitope can induce an epitope-specific response restricted to H-2^{b}, H-2^{d}, H-2^{k} and H-2^{s} in mice (Hale et al., Int. Immunol., 1:409 (1989); Berzofsky et al., J. Clin. Invest., 88:876 (1991)). Furthermore, a gain in potency for CD4 response was observed in mice when the glutamic acid residue at position 436 (E436) in the T1 epitope was replaced with alanine (A) in mice (E436A) (Boehncke et al., J. Immunol., 150:331 (1993); Ahlers et al., Proc. Natl. Acad. Sci. USA, 94:10856 (1997); Ahlers et al., J. Clin. Invest., 108:1677 (2001)), suggesting that substitution of amino acid residues that interfere with epitope binding might allow the design of a more potent vaccine.

However, this substitution of one amino acid residue enhanced binding to only one murine class II MHC molecule. Little is known about the HLA class II restriction of the T1 epitope in humans. Therefore, the identity of other amino acid-substitutions that would improve recognition with class II HLA molecules of humans is of interest in order to design a more effective immunogenic composition. The present invention provides epitope-enhanced T1 peptides that can be used in immunogenic compositions that demonstrate an improved antigen specific helper T cell (CD4⁺) response. These peptide can be used either individually or in combination with other peptides and/or an adjuvant to provide immunogenic compositions and methods for administration to patients with an HIV infection as described hereinbelow.

### BRIEF SUMMARY OF THE INVENTION

The present invention provides isolated polypeptides comprising an enhanced T1 epitope. The enhanced T1 epitopes include a peptide region corresponding to positions 428-443 of HIV IIIB gp160 protein (KQIINMWQEVGKAMYA, SEQ ID NO: 1) (corresponding to residues 421-436 in the consensus clade B HIV-1 sequence) and having at least one of the following amino acid substitutions: Val to Ile at position 437 (V437I); Lys to Arg at position 439 (K439R); Tyr to Ala at position 442 (Y442A); Tyr to Phe at position 442 (Y442F); or Tyr to Ile at position 442 (Y442I). For example, in specific embodiments, the T1 epitope has the amino acid sequence KQIINMWQEIGKAMYA (SEQ ID NO:14), KQIINMWQEVGRAMYA (SEQ ID NO:18), KQIINMWQEVGKAMAA (SEQ ID NO:22), KQIINMWQEVGKAMFA (SEQ ID NO:23), or KQIINMWQEVGKAMIA (SEQ ID NO:24). In some variations, the enhanced T1 epitope has at least two of the amino acid substitutions at different positions (e.g., the amino acid substitutions V437I, K439R, and any one of Y442A, Y442F, or Y4421). For example, in one exemplary embodiment, the enhanced T1 epitope has the amino acid substitutions V437I, K439R, and Y442A (e.g., a peptide having the sequence KQIINMWQEIGRAMAA (SEQ ID NO:25)). In yet other variations, the polypeptide of the invention comprises the amino acid sequence

| | |
|---|---|
| KQIINMWQEIGKAMYAPPISGQIR, | (SEQ ID NO:27) |
| KQIINMWQEVGRAMYAPPISGQIR, | (SEQ ID NO:28) |
| KQIINMWQEVGKAMAAPPISGQIR, | (SEQ ID NO:29) |
| or | |
| KQIINMWQEIGRAMAAPPISGQIR. | (SEQ ID NO:30) |

The polypeptide of the present invention can consist essentially of the peptide corresponding to positions 428-443 of HIV IIIB gp160 protein. Alternatively, the polypeptide can further include, for example, one or more other epitopes. For example, in some embodiments, the polypeptide includes at least one HIV cytotoxic T lymphocyte (CTL) epitope such as, e.g., p18 peptide. Suitable p18 peptides include those comprising the amino acid sequence RIQRGPGRAFVTI (SEQ ID NO:31); RIHIGPGRAFYTT (SEQ ID NO:32); SIHIGPGRAFYAT (SEQ ID NO:33); SITKGPGRVIYAT (SEQ ID NO:34); SIYIGPGRAFHTT (SEQ ID NO:35); GIAIGPGRTLYAR (SEQ ID NO:36); RVTLGPGRVWYTT (SEQ ID NO:37); SLSIGPGRAFRTR (SEQ ID NO:38); SISIGPGRAFFATTD (SEQ ID NO:39); SIRIGPGKVFTAKGG (SEQ ID NO:40); FGPGQALYTTGI (SEQ ID NO:41); STPIGLGQALYTTRG (SEQ ID NO:42); STPIGLGQALYTTRI (SEQ ID NO:43); or RTPTGLGQSLYTTRS (SEQ ID NO:44), and the like. Other useful CTL epitopes that can be combined with the epitope-enhanced T1 epitopes of the present invention include CTL epitopes from HIV-1 reverse transcriptase (RT), gp41, p17 Gag, or Nef (e.g., peptides having the amino acid sequence VIYQYMDDL (SEQ ID NO:45); ILKEPVHGV (SEQ ID NO:46); SLLNATDIAV (SEQ ID NO:47); SLYNTVATL (SEQ ID NO:48); or AFHHVAREL (SEQ ID NO:49). In yet other variations, the polypeptides as set forth above further include a neutralizing antibody epitope.

In certain aspects of the invention, pharmaceutical compositions are provided that include (a) a polypeptide comprising an enhanced T1 epitope as set forth above and (b) at least one other pharmaceutically acceptable ingredient. The pharmaceutically acceptable ingredient can be, for example, a carrier or an adjuvant.

In yet another aspect, the polypeptides comprising an enhanced T1 epitope, including, e.g., pharmaceutical compositions comprising the polypeptides, are used in methods for inducing an enhanced T1 epitope-specific immune response. The methods generally include contacting an antigen presenting cell (APC) with a polypeptide comprising an enhanced T1 epitope, whereby the enhanced T1 epitope binds to a HLA class II molecule of the APC and is presented on the surface of the APC to a T1 epitope-specific CD4⁺ T cell, thereby inducing an enhanced T1 epitope-specific immune response. In one exemplary embodiment, the CD4⁺ T cell is characterized as being HLA DR13⁺. More specifically the CD4⁺ T cell is a cell line isolated from an individual that is HLA DR13⁺, such as, for example the cell line designated KT9 as described herein.

Also provided are methods of determining the amino acid sequence of an enhanced T1 helper T cell epitope. The methods generally include (a) contacting a first HLA-DR13⁺ antigen presenting cell (APC) with a polypeptide comprising a first peptide region, said first peptide region corresponding to positions 428-443 of HIV IIIB gp160 protein and having at least one amino acid substitution relative to the amino acid sequence set forth in SEQ ID NO: 1; (b) contacting a second HLA-DR13⁺ APC with a control polypeptide comprising a second peptide region, said second peptide region having the amino acid sequence set forth in SEQ ID NO: 1; (c) contacting the first APC with a first T1 epitope specific CD4⁺ T cell; (d) contacting a second APC with a second T1 epitope specific CD4⁺ T cell; and (e) detecting for each of the first and second CD4⁺ T cells the level of T1-specific cell activation (e.g., an increase in T cell proliferation), wherein an increase in activation of the first CD4⁺ T cell relative to the second CD4⁺ T cell identifies the amino acid sequence of the first peptide region as an enhanced T1 helper T cell epitope. In a specific variation of the method, the first and second CD4⁺ T cells are KT9 cells.

### BRIEF DESCRIPTION OF THE DRAWINGS

FIGS. 1A and 1B provide characterization of the CD4⁺ cell line designated KT9. FIG. 1A depicts an inhibition assay of the KT9 cell line. Proliferation of KT9 cells in response to T1 peptide was inhibited with anti-CD4, anti-CD8, anti-HLA-DR, anti-HLA-DQ antibody or control IgG. The concentration of each antibody was 10 μg/ml. FIG. 1B depicts the level of proliferation of KT9 cells against antigen presenting cells (APCs) of different HLA-DR types partially matched with the T cell donor (DRβ1*). The haplotypes of HLA-DR used in this assay were DRβ1*(01, 13), DRβ1*(01, 11), DRβ1*(04, 13), and DRβ1*(11,13).

FIG. 2 depicts the level of proliferation of KT9 cells against T1 variant peptides with alanine-substitutions at each position. The concentration of each substituted peptide was 10 μM. Each peptide with its SEQ ID NO: designation is listed in Table 1.

FIGS. 3A and 3B provide characterization of T1 peptides having amino acid substitutions in the anchor positions. FIG. 3A depicts the level of proliferation of KT9 cells against the substituted T1 peptides. The concentration of each substituted peptide was 10 μM. Each peptide with its SEQ ID NO: designation is listed in Table 1. FIG. 3B depicts a titration of peptides designated 53 (SEQ ID NO:14), 59 (SEQ ID NO:18), 67 (SEQ ID NO:22) and the original wild type T1 peptide (SEQ ID NO: 1) in a proliferation assay of KT9 cells.

FIG. 4 depicts the level of proliferation of KT9 cells with the multiply amino acid substituted T1 peptide 70 (SEQ ID NO:25) and the original wild type T1 peptide (SEQ ID NO:1).

### DETAILED DESCRIPTION OF THE INVENTION

The present invention provides epitope-enhanced CD4 peptides that can induce a stronger antigen specific T helper cell response to HIV. Methods are also provided for determining the amino acid sequence for an epitope-enhanced CD4 peptide of the present invention. In a particular embodiment of the present invention enhanced epitope peptides comprising the CD4 helper epitope of the HIV-1 peptide designated T1 are described. The peptides have at least one amino acid residue from the T1 amino acid sequence replaced with an amino acid residue that results in an enhanced antigen specific proliferation response when the peptide is contacted with a CD4⁺ T cell line isolated from an individual exposed to an HIV antigen.

The T1 antigen was the first CD4 helper epitope discovered in the HIV-1 envelope protein (Cease et al., Proc. Natl. Acad. Sci. USA, 84:4249 (1987); Clerici et al., Nature, 339:383 (1989); Berzofsky et al., Nature, 334:706 (1988)). A CD4 response specific for this epitope could be observed in some HIV-seropositive individuals in the United States and healthy volunteers vaccinated with gp160 in Zaire, Africa (Clerici et al., Nature, 339:383 (1989); Berzofsky et al., Nature, 334:706 (1988)). These facts indicated that the T1 epitope was obviously presented by some relatively prevalent human HLA class II molecules. However, the actual class II restriction of T1-specific CD4⁺ T cells remained to be clarified.

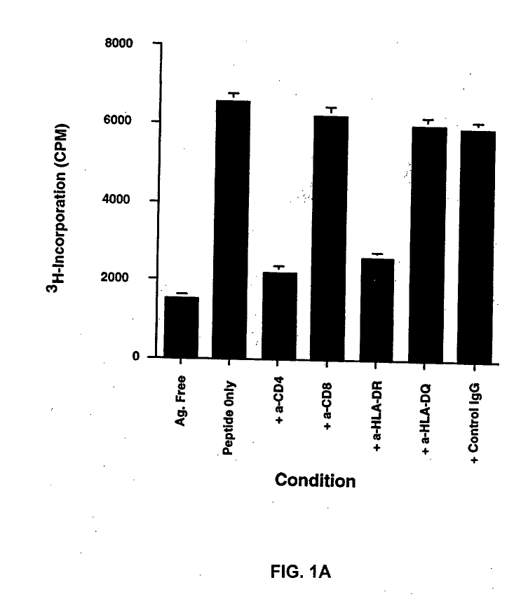

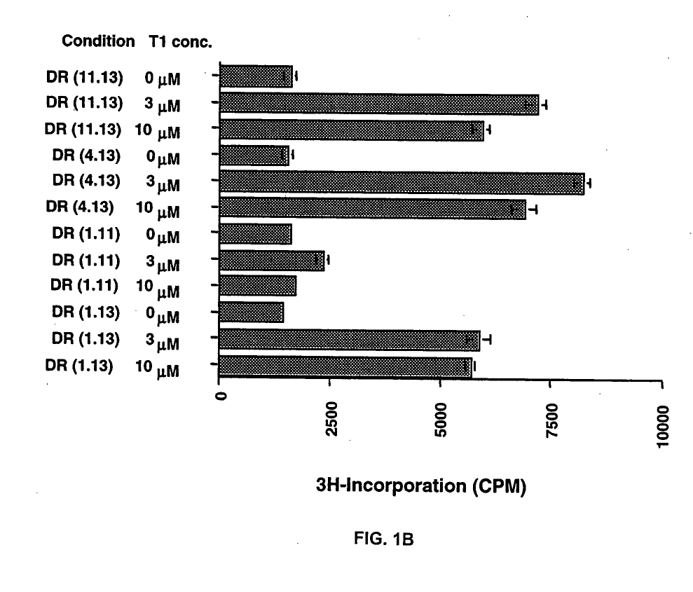

In the present invention, a T1-specific human CD4⁺ T cell line (KT9) was developed from a healthy Caucasian American volunteer immunized with a canarypox virus vector expressing HIV-1 envelope protein gp120 and this CD4 helper line was found to be restricted to DRβ1*13 (FIG. 1B). DR13 is one of the major HLA class II haplotypes found in Africa where there are more than 25 million HIV-infected patients (Mwau et al., J. Gene. Med., 5:3 (2003)), and is also common in U.S. Caucasians as well as African Americans (Verreck et al., Immunogenetics, 43:392 (1996)). Thus, the T1 antigen can be a very useful CD4 epitope as an HIV-1 immunogenic composition component for administration to individuals in North America and Africa. Because HIV epitopes may have evolved to evade the immune system, methods as disclosed in the present invention for epitope enhancement have been applied for amino acid sequence modification to increase the potency of this epitope.

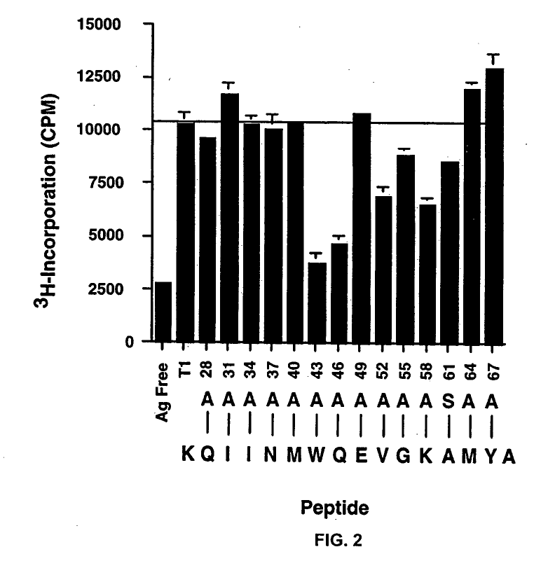

The present invention provides methods for screening for peptides that can induce a stronger CD4⁺ T cell response against the T1 epitope as measured by an increase in the proliferative response of an antigen specific CD4⁺ T cell line designated herein as KT9. Certain amino acid-substituted variants of the T1 peptide were tested for the induction of the proliferative response. It was determined that except for two cases, T1 peptides with alanine-substituted individually in each position could not induce a stronger proliferative response from KT9 cells than the original wild type T1 peptide (FIG. 2). However, the likely binding core was identified as WQEVGKAMY (SEQ ID NO:2) based on the alanine substitutions that demonstrated diminished recognition, together with proliferation assay data using truncated peptides of T1.

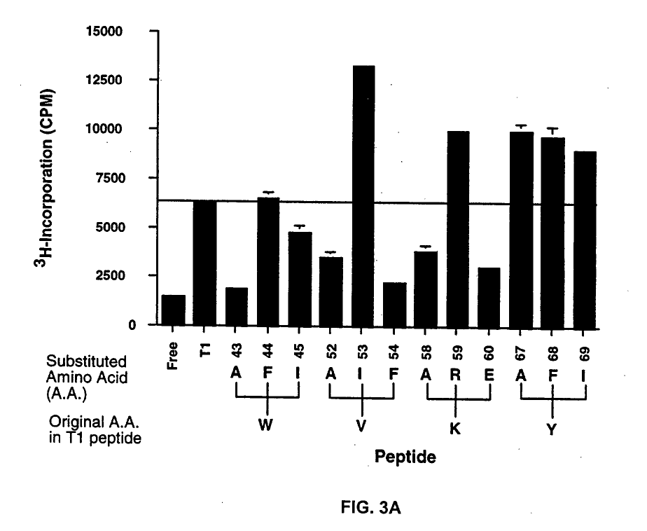

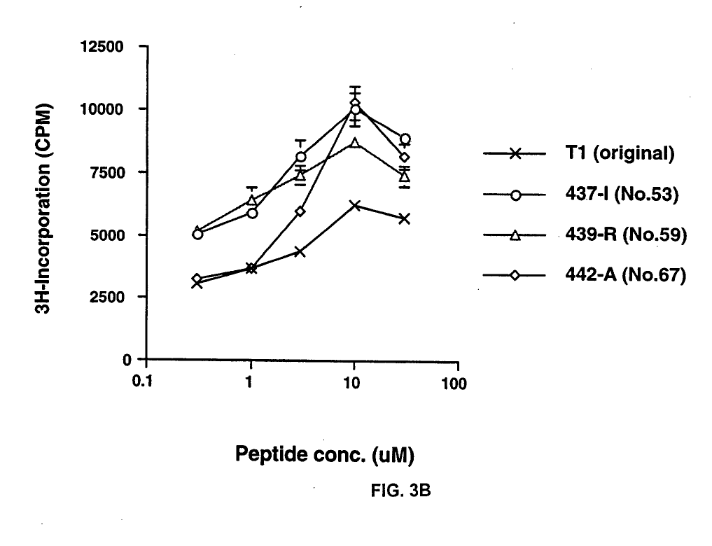

Most HLA-DR binding core sequence motifs typically have 4 anchor residues. As shown in FIGS. 3A and B, an aromatic amino acid and an aliphatic amino acid were preferentially found in position 1 and in position 4, respectively. Although the substitution of phenylalanine for tryptophan in position 1 did not produce a better CD4 response, the substitution of isoleucine for valine in the T1 peptide increased proliferation of KT9 cells up to 2-fold over the original T1-stimulated response. Both valine and isoleucine belong to the same aliphatic and branched chain amino acid group. This suggested that there might be a hierarchy for binding to pockets of HLA-DR molecules among amino acids in the same group. Position 6 is thought to be an allele-specific anchor (Hammer et al., Cell, 74:197 (1993)). In the case of DR13, a positively charged amino acid is preferable.

Also from the data in FIG. 3, the replacement of lysine (pK 10.5) by a more positively charged amino acid, e.g., arginine (pK 12.5), in position 6 enhanced the antigen specific CD4 response. The replacement by the more positively charged amino acid in this allele-specific anchor position might be a broadly applicable method for epitope enhancement of other CD4 epitopes presented by DR13.

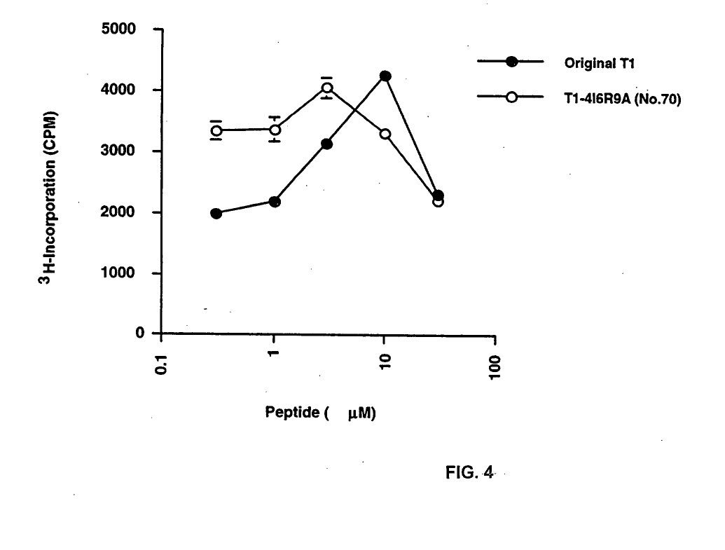

It has been previously reported that an epitope-enhanced HIV-1 CTL epitope substituted with the appropriate amino acids in all class I MHC anchor positions can induce a better CTL response in vitro and in vivo (Okazaki et al., J. Immunol., 171:2548 (2003)). Other enhanced CTL epitopes from HIV-1 reverse transcriptase were found to have improved binding to a class I molecule as described by (Pogue et al., Proc. Natl. Acad. Sci. USA, 92:8166 (1995)). However, it is equally important to determine whether the epitope-enhancement strategy could magnify the activation of human HIV specific CD4⁺ T cells recognizing a peptide binding to a human class II HLA molecule to help the expansion and maintenance for HIV specific CTLs. To date, there is little experience with epitope enhancement for peptides binding human class II HLA molecules. Thus, the present inventors have determined that this principle was true for the epitope-enhancement of this HIV CD4 epitope by first isolating a CD4⁺ cell line, e.g., the cell line described herein and designated KT9, from an individual vaccinated with an HIV-1 gp120 immunogen and using this cell line to determine amino acid substitution variations of the T1 CD4 epitope. As shown in FIG. 4, the multiply substituted peptide 70 (SEQ ID NO:25) shifted the peak proliferative response of KT9 to lower concentrations of peptide. The result demonstrated that the epitope-enhanced HIV CD4 epitope substituted with appropriate combinations of anchor residues could also induce a stronger wild-type specific CD4⁺ T cell response. Such an epitope-enhancement strategy could induce stronger CD4 responses in humans and might provide more effective CD4⁺ T cell-mediated help for CTL maintenance against HIV.

The present findings demonstrate that modifying this HIV-1 CD4 epitope with certain amino acid substitutions in the anchor regions can magnify the T1 specific CD4 helper response compared to the original T1 peptide confirming epitope enhancement for CD4⁺ epitopes. However, the T1 peptide is just one of a number of HIV CD4 epitopes. This strategy could be more effective when applied to multiple conserved CD4 epitopes in HIV, including HIV-2, as well as to HIV CTL epitopes, as recently described (Okazaki et al., J. Immunol., 171:2548 (2003)). The application of this strategy to other HIV candidate epitopes could be necessary for the development of the next generation of HIV immunogenic compositions. Also, the present studies provide a rational strategy for the construction and/or selection of enhanced epitopes that can be used to build second generation immunogenic compositions, applicable to all forms of compositions including, for example, peptide, DNA, recombinant viral or bacterial vector, or live attenuated virus compositions.

Unless indicated otherwise, the numbering of HIV-1 amino acid residues used herein is based on the original Ratner et al. numbering (see Ratner et al., Nature, 313:277-284 (1985)). The corresponding numbers of the HIV-1 consensus sequence (used by the Los Alamos database for the sequences of all strains) can be obtained by subtracting 7 from the Ratner numbering (e.g., residues 428-443 according to the Ratner numbering correspond to residues 421-436 in the consensus sequence).

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the typical methods and materials are now described. All publications mentioned herein are incorporated by reference in their entirety for all purposes. Unless mentioned otherwise, standard procedures or methodology, well-known to one of ordinary skill in the art, are followed.

### I. Enhanced T1 Epitope Polypeptides

In one aspect, the present invention provides polypeptides comprising an enhanced T1 epitope of HIV gp160. As used herein, “enhanced T1 epitope” means an HIV T1 epitope corresponding to positions 428-443 of HIV IIIB gp160 (SEQ ID NO:1) and which stimulates an enhanced T1 epitope-specific immune response as defined hereinbelow.

The term “polypeptide” refers to a polymer of amino acids and its equivalent and does not refer to a specific length of the product; thus, peptides and oligopeptides are included within the definition of a polypeptide. Also included within the definition of “polypeptide” are, for example, polypeptides containing one or more analogs of an amino acid (e.g., unnatural amino acids, and the like), polypeptides with substituted linkages as well as other modifications known in the art, both naturally and non-naturally occurring. The terms “amino acid” or “amino acid residue”, as used herein, refer to naturally occurring L amino acids or to D amino acids. The commonly used one- and three-letter abbreviations for amino acids are used herein (see, e.g., Alberts et al., Molecular Biology of the Cell, (Garland Publishing, Inc., New York, 3d. ed. (1994)).

A peptide is “corresponding” to positions 428-443 of HIV IIIB gp160 (SEQ ID NO:1) where the amino acid sequence of the peptide differs from the amino acid sequence set forth in SEQ ID NO:1 by one, two, three, or four amino acid substitutions. Regions corresponding to positions 428-443 of other HIV isolates, including isolates of HIV-2, can be determined by the comparison of the amino acid sequence of gp120 protein from the isolate of interest with the amino acid sequence of the gp120 from the HIV IIIB isolate by methods well known to the skilled artisan.

In the context of a T1-specific immune response, “enhanced” is used herein synonymously with “stronger,” “better,” “improved,” or “more substantial” and refers to an immune response comprising increased activation of T1-specific helper T lymphocytes relative to the level of T1-specific helper T cell activation observed when using the T1 peptide having SEQ ID NO: 1 as an immunogen. T cell activation can be determined by any of the various methods known in the art, including, for example, by measuring cell proliferation (e.g., ³H-thymidine proliferation assays), cytokine production (e.g., IL-2 or IFN-γ), or expression of T lymphocyte cell-surface activation markers.

In typical embodiments, the enhanced T1 epitope is a peptide corresponding to positions 428-443 of HIV IIIB gp160 (KQIINMWQEVGKAMYA; SEQ ID NO:1) and having at least one of the following amino acid substitutions:

(a) Val to Ile at position 437 (V437I);

(b) Lys to Arg at position 439 (K439R); and

(c) Tyr to Ala, Phe, or Ile at position 442 (Y442A, Y442F, or Y442I).

For example, in specific embodiments, the enhanced T1 epitope has the amino acid sequence KQIINMWQEIGKAMYA as set forth as SEQ ID NO:14, KQIINMWQEVGRAMYA as set forth as SEQ ID NO: 18, KQIINMWQEVGKAMAA as set forth as SEQ ID NO:22, KQIINMWQEVGKAMFA as set forth as SEQ ID NO:23, or KQIINMWQEVGKAMIA as set forth as SEQ ID NO:24.

Optionally, the enhanced T1 epitope has at least two of the amino acid substitutions set forth in (a), (b), and (c) above. For example, particularly suitable enhanced T1 epitopes are those having three of the above amino acid substitutions at positions 437, 439, and 442 (i.e., V437I; K439R; and any one of Y442A, Y442F, or Y4421). In one exemplary embodiment, the enhanced epitope has the amino acid substitutions V437I, K439R, Y442A (e.g., a peptide having the sequence KQIINMWQEIGRAMAA set forth as SEQ ID NO:25).

Enhanced T1 epitope polypeptides of the invention can incorporate additional epitopes (multideterminant peptides). The additional antigenic determinants can be, for example, discontinuous (i.e., comprising two or more separate peptide segments, required for immunoreactivity, from the same antigenic protein) or continuous, and can further include full-length, enhanced T1 epitope-containing proteins recognized by T lymphocytes. For example, in certain variations, polypeptides of the present invention have a length of less than 100 amino acids, less than 50 amino acids, or less than 25 amino acids.

In some variations, polypeptides of the invention are chimeric constructs that include, in addition to the enhanced T1 epitope, a second helper T cell epitope, a CTL activating epitope, and/or a neutralizing antibody epitope. Enhanced helper T cell or enhanced CTL epitopes are particularly suitable.

For example, in some embodiments, the polypeptide having an enhanced T1 epitope is a multideterminant cluster peptide or “PCLUS” peptide of HIV IIIB gp160 protein. A cluster peptide contains multiple overlapping helper T cell activating epitopes that can be presented by multiple class II HLA molecules. One example of a PCLUS peptide having a T1 epitope and suitable for enhancement according to the present invention is PCLUS 3, having the amino acid sequence KQIINMWQEVGKAMYAPPISGQIR (SEQ ID NO:26) (see U.S. patent application No. 5,939,074). For example, in certain variations, the T1 epitope of PLCUS 3 is enhanced by incorporating one or more of the following amino acid substitutions: Val to Ile at position 437; Lys to Arg at position 439; and/or Tyr to Ala, Phe, or Ile at position 442. Exemplary enhanced PCLUS 3 peptides include, e.g.,

| | |
|---|---|
| KQIINMWQEIGKAMYAPPISGQIR, | (SEQ ID NO:27) |
| KQIINMWQEVGRAMYAPPISGQIR, | (SEQ ID NO:28) |
| KQIINMWQEVGKAMAAPPISGQIR, | (SEQ ID NO:29 |
| and | |
| KQIINMWQEIGRAMAAPPISGQIR. | (SEQ ID NO:30) |

CTL epitope peptides from HIV that can be linked to an enhanced T1 epitope include, for example but not limitation, CTL epitopes based on the p18 peptide derived from the HIV-1 (IIIB) gp160 envelope glycoprotein. Various P18 peptides can be selected as peptide antigens for use with the present invention. Particularly suitable corresponding p18 peptides from different HIV-1 isolates include polypeptides comprising the following amino acid sequences, e.g., RIQRGPGRAFVTI (SEQ ID NO:31, isolate 111B); RIHIGPGRAFYTT (SEQ ID NO:32, isolate MN); SIHIGPGRAFYAT (SEQ ID NO:33, isolate SC); SITKGPGRVIYAT (SEQ ID NO:34, isolate RF); SIYIGPGPAFHTT (SEQ ID NO:35, isolate SF2); GIAIGPGRTLYAR (SEQ ID NO:36, isolate NY5); RVTLGPGRVWYTT (SEQ ID NO:37, isolate CDC4); SLSIGPGRAFRTR (SEQ ID NO:38, isolate WMJ2); SISIGPGRAFFATTD (SEQ ID NO:39, isolate Z321); SIRIGPGKVFTAKGG (SEQ ID NO:40, isolate Z3); FGPGQALYTTGI (SEQ ID NO:41, isolate MAL); STPIGLGQALYTTRG (SEQ ID NO:42, isolate Z6); STPIGLGQALYTTRI (SEQ ID NO:43, isolate JYI); and RTPTGLGQSLYTTRS (SEQ ID NO:44).

Other suitable CTL epitopes for use in accordance with the present invention include, for example, epitopes derived from HIV reverse transcriptase (e.g., the VL9 epitope VIYQYMDDL (SEQ ID NO:45), see Harrer et al., J. Infect. Dis., 173:476 (1996) or the IV9 epitope ILKEPVHGV (SEQ ID NO:46), see Walker et al., Proc. Natl. Acad. Sci. USA, 86:9514 (1989)); epitopes derived from HIV gp41 including (e.g., the SV10 epitope SLLNATDIAV (SEQ ID NO:47), see Dupuis et al., J. Immunol., 155:2232 (1995)); epitopes derived from HIV p17 Gag including (e.g., the SL9 epitope SLYNTVATL (SEQ ID NO:48), see Johnson et al., J. Immunol., 147:1512 (1991); Nixon and McMichael, AIDS, 5:1049 (1991); and epitopes derived from HIV Nef (e.g., the AL9 epitope AFHHVAREL (SEQ ID NO:49), see Brander and Goulder, In Korber et al., eds., HIV Molecular Database, (Los Alamos National Laboratory, New Mexico, IV-1-IV-17 (1999)). Additional CTL epitopes have been defined and can be found for example in the HIV Molecular Database internet pages of Los Alamos National Laboratory, Los Alamos, N. Mex.

In some variations, polypeptides having an enhanced T1 epitope are cluster peptide vaccine constructs (CLUVAC). A CLUVAC construct is a chimeric peptide comprising (a) a subregion with overlapping helper T cell epitopes (cluster peptide), (b) a subregion with a CTL activating epitope, and (c) a subregion that elicits the production of a neutralizing antibody. CLUVACs of the present invention contain an enhanced T1 epitope in the cluster peptide region (e.g., an enhanced PCLUS 3 as described supra). Exemplary CLUVACs having the wild type T1 epitope are described in, e.g., U.S. Pat. No. 5,932,218 and can be modified to enhance the T1 epitope as described herein.

Further, in other embodiments, the enhanced T1 epitope polypeptide is fused, typically by chemical or recombinant techniques well-known to those of skill in the art, to a carrier protein or peptide. Suitable carrier proteins include, for example, β-galactosidase, glutathione-S-transferase, keyhole limpet hemocyanin (KLH), bovine serum albumin (BSA), and other albumins such as ovalbumin, mouse serum albumin or rabbit serum albumin.

Enhanced T1 epitope polypeptides of the invention can also include those described above but modified for in vivo use by:

(a) chemical or recombinant DNA methods to include mammalian signal peptides (Lin et al., J. Biol. Chem., 270:14255 (1995)) or a bacterial peptide such as, for example, “penetrating” (Joliot et al., Proc. Natl. Acad. Sci. USA, 88:1864 (1991)), that will serve to direct the polypeptide across cell and cytoplasmic membranes and/or traffic it to the endoplasmic reticulum (ER) of antigen presenting cells (APC), e.g., dendritic cells, which are potent CTL inducers;

(b) addition of a translocating agent, such as for example, a biotin residue which serves to direct the polypeptides across cell membranes by virtue of its ability to bind specifically to a translocator present on the surface of cells (Chen et al., Analytical Biochem., 227:168 (1995));

(c) addition at either or both the amino- and carboxy-terminal ends, of a protease blocking agent in order to facilitate survival of the relevant polypeptide in vivo. This can be useful in those situations in which the termini tend to be degraded (“nibbled”) by proteases prior to cellular or ER uptake. Such blocking agents can include, without limitation, additional related or unrelated peptide sequences that can be attached to the amino and/or carboxy terminal residues of the polypeptide to be administered. This can be done either chemically during the synthesis of the peptide or by recombinant DNA technology. Alternatively, blocking agents such as pyroglutamic acid or other molecules known to those of average skill in the art can be attached to the amino and/or carboxy terminal residues, or the amino group at the amino terminus or carboxyl group at the carboxy terminus replaced with a different moiety. Likewise, the polypeptides can be covalently or non-covalently coupled to pharmaceutically acceptable “carrier” proteins prior to administration.

Enhanced T1 epitope polypeptides of the invention further include functionally equivalent variants of the specific polypeptide described herein. The term “functionally equivalent variant,” in the context of an enhanced T1 epitope, refers to an enhanced T1 epitope of the present invention that is modified by deletion, addition, substitution or derivatization of the amino acid residues set forth herein, in any suitable manner so long as the resulting polypeptide acts in a functional manner similar to that of the enhanced T1 epitopes described herein.

The enhanced T1 epitope polypeptides can be obtained by a variety of means. Smaller peptides (typically less than 50 to 75 amino acids long) comprising the enhanced T1 epitope can be conveniently synthesized by standard chemical methods familiar to those skilled in the art (e.g., see Creighton, Proteins: Structures and Molecular Principles, (W.H. Freeman and Co., N.Y. (1983)); Stewart and Young, Solid Phase Peptide Synthesis (Pierce Chemical Company, Rockford, Ill. (1984)). Larger peptides (longer than about 75 to 100 amino acids) comprising an enhanced T1 epitope can be produced by a number of methods including recombinant DNA technology. Typically, the polypeptides of the invention are isolated or purified for use in accordance with the methods provided herein. The term “isolated” or “purified” refers to a polypeptide that has been removed from its natural cellular environment. Isolated polypeptide include, e.g., recombinant polypeptides removed from one or more components of the recombinant cellular environment in which it is produced as well as those polypeptides produced synthetically removed from one or more components of the synthetic reaction environment.

When choosing a polypeptide of the present invention to synthesize and use according to the methods provided herein, the sequence of the polypeptide may be designed to render it more soluble. Also, it is desirable that the polypeptide sequence be one that is easily synthesized, that is, a sequence that lacks highly reactive side groups. Furthermore, as indicated supra, the peptide need not be the minimal peptide that will bind to the HLA class II molecule, as longer sequences will be processed and presented sufficiently to elicit the enhanced, primary T-cell response.

Purified polypeptides having an enhanced T1 epitope can be chemically synthesized by solid phase synthesis and purified away from the other products of the chemical reactions, for example by HPLC. Alternatively, the polypeptide may be produced by the expression of a DNA sequence included in a vector in a recombinant cell. In this method of producing the peptide, purification may be accomplished by a variety of appropriate techniques well known in the art.

A fusion protein comprising an enhanced T1 epitope of the present invention produced by recombinant methods can be readily purified by utilizing an antibody or a ligand that specifically binds to the fusion protein being expressed. For example, a system described by Janknecht et al. (Proc. Natl. Acad. Sci. USA, 88:8972 (1991)) allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers. If desired, the histidine tag can be selectively cleaved with an appropriate enzyme.

It is also recognized by those skill in the present art that peptide mimetics which possess the same structure as the enhanced T1 epitope of the present invention can be useful. A peptide mimetic is a compound which has sufficient structural similarity to a peptide so that the desirable properties of the peptide are retained by the mimetic. For example, peptide mimetics used as protease inhibitors are described in, for example, WO 94/05639. A peptide mimetic refers to any peptide or non-peptide compound that is able to mimic the biological action of a naturally occurring peptide, often because the mimetic has a basic structure that mimics the basic structure of the peptide and/or has the salient biological properties of the peptide. Mimetics can include, but are not limited to, peptides that have substantial modifications from the prototype such that no side chain similarity with the peptide (such modification, for example, may decrease its susceptibility to degradation); non-proteinaceous portions of an isolated peptide; or synthetic or natural organic molecules, including nucleic acids and agents identified through combinatorial chemistry. For example, such mimetics can be designed, selected, and/or otherwise identified using a variety of methods known in the art, including, for example, construction and screening of large chemically diverse molecular libraries, libraries of synthetic or natural compound libraries, or by rational, directed or random design. The general goal of screening such libraries is to utilize sequential application of combinatorial selection to obtain high affinity agents for the binding site of interest. For directed or rational drug design the structure of the enhanced T1 peptide epitopes of the present invention can be used as a base for selection and design of a peptide mimetic.

### II. Methods of Identifying Enhanced T1 Epitopes

In another aspect of the invention, methods for determining the amino acid sequence of an enhanced helper T cell epitope are provided. The methods generally include contacting an antigen presenting cell (e.g., a dendritic cell) of a known HLA class II haplotype type (e.g., HLA-DR13⁺) with a polypeptide having a peptide region corresponding to positions 428-443 of HIV IIIB gp160 protein and having at least one amino acid substitution relative to the amino acid sequence set forth in SEQ ID NO: 1, whereby the APC presents the peptide corresponding to positions 428-443 of HIV IIIB gp160 on the APC cell surface bound to the HLA class II molecule of interest; culturing the APC in the presence of a T1 epitope specific CD4⁺ T cell, whereby the HLA-bound peptide on the APC cell surface contacts the CD4⁺ T cell; and assaying the CD4⁺ T cell for the level of T cell activation in response to presentation of peptide. The level of activation is compared with that determined for a control (wild type) peptide having SEQ ID NO: 1 (i.e., not having the amino acid substitution(s)). T cell activation is assayed using any of various known methods, including, for example, by measuring T cell proliferation, production of cytokines (such as, e.g., IL-2 or IFN-γ), or expression of T cell activation markers on the cell surface. An increase in the level of T cell activation in response to the peptide region having the amino acid substitution(s), as compared to the control peptide region, identifies the amino acid sequence of the substituted peptide region as an enhanced T1 helper T cell epitope. In certain variations of the method, a T1-specific T cell line is used. The CD4⁺ DR13⁺ T cell line designated KT9 described hereinbelow is particularly suitable.

### III. Methods of Inducing an Enhanced T1 Epitope Specific Immune Response

In yet another aspect, polypeptides having an enhanced T1 epitope, as described supra, are used in methods of inducing an enhanced T1-specific immune response. The methods generally include contacting a cell population comprising an HLA-DR13⁺ antigen presenting cell with an enhanced T1 epitope polypeptide, whereby the enhanced T1 epitope binds to the HLA-DR13 molecule of the APC and is presented on the surface of the APC to a T1 epitope-specific CD4⁺ T cell, thereby inducing an enhanced T1 epitope-specific immune response. Enhancement of a T1-specific immune response can include, for example, increased T cell proliferation, increased cytokine production, or increased expression of specific cell surface molecules associated with T cell activation (e.g., costimulatory molecules), where the increase is relative to T1-specific immune responses induced using a non-enhanced (wild type) T1 epitope (e.g., a peptide having the sequence set forth in SEQ ID NO:1). Enhanced activation of T1-specific helper T cells is useful, for example, for augmenting B cell activation in response to neutralizing antibody epitopes of HIV proteins and/or augmenting cytotoxic T lymphocyte (CTL) activation in response to CTL epitopes of HIV proteins. Augmentation of HIV epitope-specific B cell and CTL responses is advantageous for enhancement of the humoral or cell-mediated immune responses against HIV.

The methods can be used to enhance T1-specific immune responses in vitro, in vivo, or ex vivo. In vitro methods will include culture systems having one or more HLA-DR⁺ antigen presenting cells and one or more T1 epitope-specific CD4⁺ T cells. In one exemplary embodiment, the T1-specific T cell is the CD4⁺ T cell line designated KT9. Also, in certain variations, the methods further include maintaining the APC and CD4⁺ T cell in the presence of one or more cells of other immune cell-type(s) such as, e.g., B cells or CTLs. In these embodiments, one or more HIV neutralizing antibody epitopes or CTL epitopes specifically recognized by the B cell or CTL are optionally introduced into the culture system, thereby augmenting HIV epitope-specific B cell or CTL activation in the presence of an enhanced T1-specific CD4⁺ T cell response.

For enhancement of an anti-HIV immune response in vivo, an enhanced T1 epitope polypeptide is typically administered to a subject, particularly a subject having a HLA-DR13 haplotype, as a pharmaceutical composition in the form of a polypeptide solution comprising a pharmaceutically acceptable carrier. “Pharmaceutically acceptable carrier,” as used herein, refers to any biologically compatible vehicle which is suitable for administration to an animal (e.g., physiological saline). Standard methods for delivery of polypeptides can be used (e.g., packaging in liposomes for intracellular delivery). Such methods are well known to those of ordinary skill in the art. It is expected that an intravenous dosage of approximately 1 to 100 moles of the polypeptide of the invention would be administered per kg of body weight per day. The compositions of the invention are useful for parenteral administration, such as intravenous, subcutaneous, intramuscular, intraperitoneal, transdermal, transmucosal, oral, nasal, rectal, urogenital, and the like. For example, a unit dose of the peptide ranges from 0.1 to 100 mg, which may be administered, one time or repeatedly, to a subject. In certain embodiments, the enhanced T1 epitope polypeptide composition is administered alone. In other variations, the polypeptide composition is administered together (simultaneously or sequentially) with other active agents (e.g., other immunogenic compositions, cytotoxic drugs, anti-viral, anti-bacterial, protease inhibitors, as an adjuvant to other therapeutic modalities, and the like.).

Polypeptide solutions are optionally lyophilized or granulated with a vehicle such as a sugar. When the compositions are administered by injection, they are typically dissolved in, for example, distilled water or another pharmaceutically acceptable excipient prior to the injection. In typical variations, the composition further includes a pharmaceutically acceptable adjuvant. “Adjuvant,” as used herein, means an agent which enhances the immunogenicity of a polypeptide having one or more antigenic determinants when administered with the polypeptide, but which does not induce an immune response when administered alone. QS-21, incomplete Freund's adjuvant, and aluminum hydroxide (alum) for example are typical adjuvants for use in accordance with the present methods. Many other adjuvants appropriate for use in particular animal species, including humans, are well known in the art as are methods for assisting in the selection of a particular adjuvant to prepare a pharmaceutical composition of the present invention.

In yet other embodiments, ex vivo procedures are used. For example, lymphocytes (e.g., derived from peripheral blood mononuclear cells, lymph nodes, cord blood, and the like) are removed from a subject, cultured with HLA-DR⁺ APCs contacted with one or more polypeptides of the invention, and returned to the subject. Typically, the APCs are syngeneic to a HLA-DR⁺ subject (e.g., autologous APCs that have also been removed from the HLA-DR⁺ subject or an HLA-matched donor). In other variations, a substantially pure population of APCs is isolated from a HLA-DR13⁺ subject, contacted with an enhanced T1 epitope polypeptide of the present invention, and then re-administered to the subject, thereby presenting the enhanced T1 antigen to helper T cells in the subject in vivo. For example, the peptide is added to 10⁷ to 10⁹ APCs (e.g., dendritic cells) originated from a subject at a final concentration of 0.1 to 10 μM in culture medium, the cells are then cultivated for several hours to one day, or more, and thereafter are intravenously administered to the subject.

In other variations, the enhanced T1 epitope polypeptides are used together with CTL HIV epitope peptides (e.g., linked to the T1 epitope in a chimeric construct as described supra, or as separate constructs) to induce enhanced CTL responses against HIV. For ex vivo embodiments, for example, immune cells are removed from a subject and cultured with the peptides comprising the CTL and enhanced T1 epitopes before readministering the cells to the subject. Optionally, the cells are continuously cultivated in vitro in a culture medium to which recombinant interleukin 2 has also been added; following culture of the cells over several weeks to induce CTL, activated CTL are then intravenously injected into the subject.

### IV. Methods for Diagnosis of HIV-1 Infection of Patients Using Enhanced T1 Epitope Polypeptides

In another aspect, enhanced T1 epitope polypeptides are utilized for the diagnosis of HIV-1 exposure and/or infection in patients. Methods of diagnosis generally comprise isolation of a population of cells comprising T lymphocytes and APCs from the patient, contacting the cells with an enhanced T1 epitope polypeptide, and detection of a HIV-1 gp160-specific T cell response to the polypeptide. The detection of HIV-1 gp160-specific T cell responses to the enhanced T1 epitope polypeptide can be accomplished, e.g., by standard techniques of T cell proliferation and production of IL-2 or other lymphokines (see, e.g., Clerici et al., Nature, 339:383-385 (1989)). In alternative embodiments, the diagnostic assay is, e.g., of a standard cytotoxicity format.

### EXAMPLES

In this example a CD4⁺ T cell line was isolated from a healthy donor previously immunized with a canarypox virus vector expressing HIV-1 gp120. The cell line was characterized and used to screen T1 peptides comprising various amino acid substitutions for their ability to stimulate T cell activation. The first peptides tested comprised single amino acid substitutions in each position of the T1 peptide. The ability of each of the amino acid substituted peptides stimulate T cell activation was used to define the region of the T1 peptide comprising the HLA class II binding site. Based on a general understanding of the amino acid characteristics of the anchor residues of an HLA class II binding site various amino acid substitutions were carried out in the anchor residue positions. T1 peptides demonstrating enhanced T cell proliferation activity were selected. The materials and methods for this example are set forth briefly below:

Synthetic peptides. Peptides were prepared in an automated multiple peptide synthesizer (Symphony; Protein Technologies, Inc., Tucson, Ariz.) using Fmoc chemistry. The peptides were purified by reverse-phase HPLC, and their sequences were confirmed where needed on an automated sequencer (477A; Applied Biosystems, Foster City, Calif.) or by amino acid analysis.

Antigen Presenting Cell preparation. After thawing frozen stocks of PBMCs from the donor, 5×10⁶ PBMCs were cultured with 1000 U/ml hGM-CSF and 1000 U/ml hIL-4 in a 6-well culture plate in RPMI 1640 medium containing 10% autologous serum. Human granulocyte-macrophage-colony stimulating factor (hGM-CSF) and human Interleukin 4 (hIL-4) were added every other day to grow out dendritic cells. After pulsing with an appropriate concentration of peptide, soluble CD40-ligand (CD40-L, gp39) was added to the culture at day 4 to mature the dendritic cells. The next day, after being harvested, the cells were irradiated with 10,000 rad and washed three times, the cultured cells enriched in monocyte-derived dendritic cells, were used as an antigen presenting cell (APC) source for T cell culture development and maintenance. Peptide pulsing was not done in the preparation for assays.

Generation of primary peptide-specific CD4⁺ T cell line. 5×10⁵/well freshly isolated PBMCs obtained from the healthy donor immunized with a canarypox virus vector expressing HIV-1 gp120 were cultured with 3 μM T1 peptide (SEQ ID NO:1) in 96-well U-bottom plates in R2E (RPMI 1640: Eagles/Hanks Amino Acid media=1:1) medium containing 10% autologous plasma. On day 7, 1×10⁶ cultured cells were re-stimulated with 8×10⁴ irradiated APCs pulsed with 0.01 μM T1 peptide (SEQ ID NO:1) in a 48-well culture plate. One day after each restimulation, T cell growth factor (Zeptometrix, Buffalo, N.Y.) was added to a final concentration of 10%. Cell lines were maintained in 10% autologous plasma R2E medium containing 1 μM sodium pyruvate, nonessential amino acids (Biofluid, Rockville, Md.), 4 μM glutamine, 100 U/ml penicillin, 100 μg/ml streptomycin, and 50 μM 2-mercaptoethanol. After checking the antigen specific response, the cell line designated KT9 was restimulated every other week for maintenance.

Proliferation assay. T cell proliferation was assessed by culturing 5×10⁴ KT9 cells with 1×10⁴ autologous APCs with or without antigenic peptide in 200 μl of R2E medium containing 10% autologous serum in 96-well U-bottomed culture plates for 3 days. Cultures were pulsed with 1 μCi of ³H-Thymidine/well for the last 24 h. Plates were harvested and counted in a 1205 Betaplate counter (Wallac, Turku, Finland). All samples were analyzed in triplicate.

### Results

The T1 specific CD4 T cell line is restricted to HLA DR 13. A peptide T1-specific CD4⁺ T cell line (KT9) was developed from a healthy volunteer immunized with a canarypox virus vector expressing gp120 of HIV-1 as described above. The cells of the KT9 cell line were characterized and determined to be CD4⁺ and HLA-DR restricted in an inhibition assay using anti specific to CD4 or HLA-DR, respectively (FIG. 1A). The HLA-DR haplotype of the volunteer was DRβ*01 and DRβ1*13. Thus, the HLA-DR-restriction of this helper line was tested using APCs from the PBMCs of different volunteers sharing one or the other HLA-DR type. As shown in FIG. 1B, the KT9 helper line was restricted to DRβ1*13 which is commonly found in US Caucasians and is also one of the major HLA-DR haplotypes in Africa (Sintasath, Hum. Immunol., 60:1001 (1999); Verreck et al., Immunogenetics, 43:392 (1996)).

Recognition Specificity of KT9 Cells Using Single Alanine-Substituted Analogues. In a set of experiments to define key functional residues in the T1 peptide (SEQ ID NO:1), peptides with alanine substitutions at each one of the positions (or Ser where Ala was the natural residue) were synthesized (Table 1) and tested in an epitope-specific proliferation assay (FIG. 2). Among the 14 alanine or serine-substituted T1 peptides, substitutions at the first 5 positions (Peptides 28 (SEQ ID NO:3), 31 (SEQ ID NO:4), 34 (SEQ ID NO:5), 37 (SEQ ID NO:6) and 40 (SEQ ID NO:7)) did not have any effect on the peptide T1-specific CD4 response. However, the next 6 substitution peptides (Peptide 43 (SEQ ID NO:8), Peptide 46 (SEQ ID NO:11), Peptide 52 (SEQ ID NO:13), Peptide 55 (SEQ ID NO:16, Peptide 58 (SEQ ID NO:17) and Peptide 61 (SEQ ID NO:20), with the exception of Peptide 49 (SEQ ID NO: 12)) induced a significantly lower response. Furthermore, two alanine-substituted T1 peptides (Peptide 64 (SEQ ID NO:21) and Peptide 67 (SEQ ID NO:22)) demonstrated a slightly better response than the original peptide. Based on the data as depicted in FIG. 2 and of the peptide sequence motif pattern of DRβ1*13-restricted peptides (Verreck et al., Immunogenetics, 43:392 (1996)), it was hypothesized that the binding core motif of the T1 peptide (SEQ ID NO: 1) in humans was (WQEVGKAMY, SEQ ID NO:2).

TABLE 1
Peptides used in T1 epitope enhancement study*

| | 428 | 429 | 430 | 431 | 432 | 433 | 434 | 435 | 436 | 437 | 438 | 439 | 440 | 441 | 442 | 443 |
|---|---|---|---|---|---|---|---|---|---|---|---|---|---|---|---|---|
| T1 | K | Q | I | I | N | M | W | Q | E | V | G | K | A | M | Y | A |
| 28 | - | A | - | - | - | - | - | - | - | - | - | - | - | - | - | - |
| 31 | - | - | A | - | - | - | - | - | - | - | - | - | - | - | - | - |
| 34 | - | - | - | A | - | - | - | - | - | - | - | - | - | - | - | - |
| 37 | - | - | - | - | A | - | - | - | - | - | - | - | - | - | - | - |
| 40 | - | - | - | - | - | A | - | - | - | - | - | - | - | - | - | - |
| 43 | - | - | - | - | - | - | A | - | - | - | - | - | - | - | - | - |
| 44 | - | - | - | - | - | - | F | - | - | - | - | - | - | - | - | - |
| 45 | - | - | - | - | - | - | I | - | - | - | - | - | - | - | - | - |
| 46 | - | - | - | - | - | - | - | A | - | - | - | - | - | - | - | - |
| 49 | - | - | - | - | - | - | - | - | A | - | - | - | - | - | - | - |
| 52 | - | - | - | - | - | - | - | - | - | A | - | - | - | - | - | - |
| 53 | - | - | - | - | - | - | - | - | - | I | - | - | - | - | - | - |
| 54 | - | - | - | - | - | - | - | - | - | F | - | - | - | - | - | - |
| 55 | - | - | - | - | - | - | - | - | - | - | A | - | - | - | - | - |
| 58 | - | - | - | - | - | - | - | - | - | - | - | A | - | - | - | - |
| 59 | - | - | - | - | - | - | - | - | - | - | - | R | - | - | - | - |
| 60 | - | - | - | - | - | - | - | - | - | - | - | E | - | - | - | - |
| 61 | - | - | - | - | - | - | - | - | - | - | - | - | S | - | - | - |
| 64 | - | - | - | - | - | - | - | - | - | - | - | - | - | A | - | - |
| 67 | - | - | - | - | - | - | - | - | - | - | - | - | - | - | A | - |
| 68 | - | - | - | - | - | - | - | - | - | - | - | - | - | - | F | - |
| 69 | - | - | - | - | - | - | - | - | - | - | - | - | - | - | I | - |
| 70 | - | - | - | - | - | - | - | - | - | I | - | R | - | - | A | - |

*The sequences of T1 and the various substituted analogs are given in single letter amino acid code.
Numbers at the top of each column refer to positions in the HIV-1 IIIB gp160 protein.
Dashes in each row indicate that the residue at that position in that peptide is the same as T1.

Recognition Property of KT9 Cells Using Substituted Peptides in Anchor Positions. In general, a typical HLA class II binding peptide has 4 anchor amino acid residues within a span of 9 residues in positions 1, 4, 6 and 9 from the N-terminal end. In the case of the peptide sequences presented by DR β1*13, hydrophobic amino acid residues are preferentially found at position 1 and 4. A positively charged amino acid residue is frequently present at position 6 and a small or hydrophobic amino acid residue is found at position 9 (Verreck et al., Immunogenetics, 43:392 (1996)). Thus, the characteristics of the amino acid fitted to each anchor position of the postulated peptide T1 binding core peptide motif was next investigated using peptides substituted with amino acids having various chemical properties. First, peptides with a single amino acid substituted in each anchor position were used (FIG. 3A). In position 1 (tryptophan in the postulated T1 core peptide), alanine (small) and isoleucine (aliphatic)-substituted peptide T1 made the response worse. However, a phenylalanine (aromatic)-substitution did not reduce the response. This result suggested that an aromatic amino acid was required in position 1. In position 4, the alanine and phenylalanine-substituted T1 peptide elicited a poor proliferative response from KT9 cells. However, the isoleucine-substitution induced a much better helper response. This result suggested amino acid position 4 as an anchor should be an aliphatic amino acid and replacement with isoleucine could enhance the antigen specific response of the T1-specific CD4⁺ T cell line. In contrast, in anchor position 6 only a positively charged amino acid-substituted peptide induced a better T1-specific response. The more positively charged amino acid at position 6 might provide better binding to HLA-DRβ1*13 because arginine (R) has a higher pK (12.5) than lysine (K) (pK 10.5). In anchor position 9, all peptides substituted with small, aromatic or aliphatic amino acids induced a better CD4 response than the original peptide. Also in a titration assay, KT9 cells stimulated with the substituted peptides which induced the best response in each anchor position at 10 μM demonstrated a better response than with the original peptide (FIG. 3B). This result indicated that a naturally occurring CD4 epitope does not always have the optimal amino acid residue in each anchor position required for both binding to the HLA class II molecule and recognition by a CD4⁺ T cell. At the same time, it suggested that the peptides with a combination of substitutions in anchor positions might be able to induce a more substantial CD4⁺ T cell response as a vaccine construction.

Antigenic Potency of the Multiply Substituted T1 Peptide. As shown in FIG. 3B, each of three separate point mutations of the T1 peptide, Val (V) to Ile (I) in position 4 (SEQ ID NO:10), Lys (K) to Arg (R) in position 6 (SEQ ID NO:18) and Tyr (Y) to Ala (A) in position 9 (SEQ ID NO:22), respectively, enhanced the proliferative response of KT9 cells. Thus, all three mutations were combined in the peptide 70 (Table 1; SEQ ID NO:25) and the peptide was tested for antigenic potency of the multiply substituted peptide in the proliferation assay (FIG. 4). The peptide concentration in which KT9 cells showed the peak proliferative response was shifted to a lower peptide concentration when using this triple combinational peptide than the original peptide T1, and the activity remained substantially higher than that of the wild type peptide at a 10-fold lower concentration. This result suggested that the epitope-enhanced peptide that has the best binding capacity in each pocket in the HLA-molecule could induce the best response of the wild-type-specific CD4⁺ T cell. Also, the multiple substitutions in anchor positions did not interfere with recognition by the wild-type-specific human CD4⁺ T cell line.

1. An isolated polypeptide comprising an enhanced T1 epitope, said enhanced T1 epitope comprising a peptide region corresponding to positions 428-443 of HIV IIIB gp160 protein (KQIINMWQEVGKAMYA, SEQ ID NO: 1) (corresponding to residues 421-436 in the consensus clade B HIV-1 sequence) and having the following amino acid substitutions:
(a) Val to Ile at position 437 (V437I);
(b) Lys to Arg at position 439 (K439R); and
(c) an amino acid substitution selected from the group consisting of
Tyr to Ala at position 442 (Y442A);
Tyr to Phe at position 442 (Y442F); and
Tyr to Ile at position 442 (Y442I).

2-4. (canceled)

5. The polypeptide of claim 1, wherein the enhanced T1 epitope has the amino acid substitutions V437I, K439R, and Y442A.

6. The polypeptide of claim 5, wherein the enhanced T1 epitope has the amino acid sequence KQIINMWQEIGRAMAA (SEQ ID NO:25).

7-31. (canceled)

32. A method for inducing an enhanced T1 epitope-specific immune response, the method comprising:
contacting an HLA-DR13⁺ antigen presenting cell (APC) with a polypeptide comprising an enhanced T1 epitope, said enhanced T1 epitope comprising a peptide region corresponding to positions 428-443 of HIV IIB gp160 protein (KQIINMWQEVGKAMYA, SEQ ID NO:1) (corresponding to residues 421-436 in the consensus clade B HIV-1 sequence) and having at least one amino acid substitution comprising
Val to Ile at position 437 (V437I),
Lys to Arg at position 439 (K439R),
Tyr to Ala at position 442 (Y442A),
Tyr to Phe at position 442 (Y442F), or
Tyr to Ile at position 442 (Y442I);
whereby the enhanced T1 epitope binds to a HLA-DR13 molecule of the APC and is presented on the surface of the APC to a T1 epitope-specific CD4⁺ T cell, thereby inducing an enhanced T1 epitope-specific immune response.

33. The method of claim 32, wherein the T1 epitope has the amino acid sequence KQIINMWQEIGKAMYA (SEQ ID NO:14), KQIINMWQEVGRAMYA (SEQ ID NO:18), KQIINMWQEVGKAMAA (SEQ ID NO:22), KQIINMWQEVGKAMFA (SEQ ID NO:23), or KQIINMWQEVGKAMIA (SEQ ID NO:24).

34. The method of claim 32, wherein the enhanced T1 epitope has at least two of the amino acid substitutions at different positions.

35. The method of claim 34, wherein the enhanced T1 epitope has the amino acid substitutions V437I, K439R, and any one of Y442A, Y442F, or Y442I.

36. The method of claim 35, wherein the enhanced T1 epitope has the amino acid substitutions V437I, K439R, and Y442A.

37. The method of claim 36, wherein the enhanced T1 epitope has the amino acid sequence KQIINMWQEIGRAMAA (SEQ ID NO:25).

38. The method of claim 32, wherein the polypeptide comprises the amino acid sequence KQIINMWQEIGKAMYAPPISGQIR (SEQ ID NO:27), KQIINMWQEVGRAMYAPPISGQIR (SEQ ID NO:28), KQIINMWQEVGKAMAAPPISGQIR (SEQ ID NO:29), or KQIINMWQEIGRAMAAPPISGQIR (SEQ ID NO:30).

39. The method of claim 32, wherein the polypeptide consists essentially of the peptide corresponding to positions 428-443 of HIV IIB gp160 protein.

40. The method of claim 32, wherein the polypeptide further comprises an HIV cytotoxic T lymphocyte (CTL) epitope.

41. The method of claim 40, wherein the CTL epitope is a p18 peptide.

42. The method of claim 41, wherein the p18 peptide has the amino acid sequence RIQRGPGRAFVTI (SEQ ID NO:31); RIHIGPGRAFYTT (SEQ ID NO:32); SIHIGPGRAFYAT (SEQ ID NO:33); SITKGPGRVIYAT (SEQ ID NO:34); SIYIGPGRAFHTT (SEQ ID NO:35); GIAIGPGRTLYAR (SEQ ID NO:36); RVTLGPGRVWYTT (SEQ ID NO:37); SLSIGPGRAFRTR (SEQ ID NO:38); SISIGPGRAFFATTD (SEQ ID NO:39); SIRIGPGKVFTAKGG (SEQ ID NO:40); FGPGQALYTTGI (SEQ ID NO:41); STPIGLGQALYTTRG (SEQ ID NO:42); STPIGLGQALYTTRI (SEQ ID NO:43); or RTPTGLGQSLYTTRS (SEQ ID NO:44).

43. The method of claim 40, wherein the CTL epitope is an epitope from HIV reverse transcriptase (RT), gp41, gp120, p17 Gag, Nef, or Tat.

44. The method of claim 43, wherein the CTL epitope is a peptide having the amino acid sequence VIYQYMDDL (SEQ ID NO:45); ILKEPVHGV (SEQ ID NO:46); SLLNATDIAV (SEQ ID NO:47); SLYNTVATL (SEQ ID NO:48); AFHHVAREL (SEQ ID NO:49).

45. The method of claim 32, wherein the polypeptide further comprises a neutralizing antibody epitope.

46. The method of claim 32, wherein the CD4⁺ T cell is a KT9 cell.

47. A method of determining the amino acid sequence of an enhanced T1 helper T cell epitope, the method comprising:
(a) contacting a first HLA-DR13⁺ antigen present cell (APC) with a polypeptide comprising a first peptide region, said first peptide region corresponding to positions 428-443 of HIV IIIB gp160 protein and having at least one amino acid substitution relative to the amino acid sequence KQIINMWQEVGKAMYA (SEQ ID NO: 1) (corresponding to residues 421-436 in the consensus clade B HIV-1 sequence);
(b) contacting a second HLA-DR13⁺ APC with a control polypeptide comprising a second peptide region, said second peptide region having the amino acid sequence KQIINMWQEVGKAMYA (SEQ ID NO:1);
(c) contacting the first APC with a first T1 epitope specific CD4⁺ T cell;
(d) contacting a second APC with a second T1 epitope specific CD4⁺ T cell; and
(e) detecting for each of the first and second CD4⁺ T cells the level of T1-specific cell activation, wherein an increase in activation of the first CD4⁺ T cell relative to the second CD4⁺ T cell identifies the amino acid sequence of the first peptide region as an enhanced T1 helper T cell epitope.

48. The method of claim 47, wherein the first and second CD4⁺ T cells are KT9 cells.

49. The method of claim 47, wherein the increase in T cell activation is an increase in T cell proliferation.

50. A method for inducing an enhanced T1 epitope-specific immune response in a human subject having an HLA-DR13 haplotype, the method comprising:
administering to the human subject having the HLA-DR13 haplotype a polypeptide comprising an enhanced T1 epitope, said enhanced T1 epitope comprising a peptide region corresponding to positions 428-443 of HIV IIIB gp160 protein (KQIINMWQEVGKAMYA, SEQ ID NO: 1) (corresponding to residues 421-436 in the consensus clade B HIV-1 sequence) and having at least one amino acid substitution comprising
Val to Ile at position 437 (V437I),
Lys to Arg at position 439 (K439R),
Tyr to Ala at position 442 (Y442A),
Tyr to Phe at position 442 (Y442F), or
Tyr to Ile at position 442 (Y442I).

51. The method of claim 50, wherein the T1 epitope has the amino acid sequence KQIINMWQEIGKAMYA (SEQ ID NO:14), KQIINMWQEVGRAMYA (SEQ ID NO:18), KQIINMWQEVGKAMAA (SEQ ID NO:22), KQIINMWQEVGKAMFA (SEQ ID NO:23), or KQIINMWQEVGKAMIA (SEQ ID NO:24).


SEQUENCE LISTING (49 sequences)

SEQ ID NO: 1 - protein, 16 aa, Artificial Sequence
  synthetic T1 epitope peptide from HIV IIIB gp160 protein
KQIINMWQEVGKAMYA

SEQ ID NO: 2 - protein, 9 aa, Artificial Sequence
  synthetic T1 peptide binding core
WQEVGKAMY

SEQ ID NO: 3 - protein, 16 aa, Artificial Sequence
  synthetic T1 epitope peptide
KAIINMWQEVGKAMYA

SEQ ID NO: 4 - protein, 16 aa, Artificial Sequence
  synthetic T1 epitope peptide
KQAINMWQEVGKAMYA

SEQ ID NO: 5 - protein, 16 aa, Artificial Sequence
  synthetic T1 epitope peptide
KQIANMWQEVGKAMYA

SEQ ID NO: 6 - protein, 16 aa, Artificial Sequence
  synthetic T1 epitope peptide
KQIIAMWQEVGKAMYA

SEQ ID NO: 7 - protein, 16 aa, Artificial Sequence
  synthetic T1 epitope peptide
KQIINAWQEVGKAMYA

SEQ ID NO: 8 - protein, 16 aa, Artificial Sequence
  synthetic T1 epitope peptide
KQIINMAQEVGKAMYA

SEQ ID NO: 9 - protein, 16 aa, Artificial Sequence
  synthetic T1 epitope peptide
KQIINMFQEVGKAMYA

SEQ ID NO: 10 - protein, 16 aa, Artificial Sequence
  synthetic T1 epitope peptide
KQIINMIQEVGKAMYA

SEQ ID NO: 11 - protein, 16 aa, Artificial Sequence
  synthetic T1 epitope peptide
KQIINMWAEVGKAMYA

SEQ ID NO: 12 - protein, 16 aa, Artificial Sequence
  synthetic T1 epitope peptide
KQIINMWQAVGKAMYA

SEQ ID NO: 13 - protein, 16 aa, Artificial Sequence
  synthetic T1 epitope peptide
KQIINMWQEAGKAMYA

SEQ ID NO: 14 - protein, 16 aa, Artificial Sequence
  synthetic T1 epitope peptide
KQIINMWQEIGKAMYA

SEQ ID NO: 15 - protein, 16 aa, Artificial Sequence
  synthetic T1 epitope peptide
KQIINMWQEFGKAMYA

SEQ ID NO: 16 - protein, 16 aa, Artificial Sequence
  synthetic T1 epitope peptide
KQIINMWQEVAKAMYA

SEQ ID NO: 17 - protein, 16 aa, Artificial Sequence
  synthetic T1 epitope peptide
KQIINMWQEVGAAMYA

SEQ ID NO: 18 - protein, 16 aa, Artificial Sequence
  synthetic T1 peptide
KQIINMWQEVGRAMYA

SEQ ID NO: 19 - protein, 16 aa, Artificial Sequence
  synthetic T1 epitope peptide
KQIINMWQEVGEAMYA

SEQ ID NO: 20 - protein, 16 aa, Artificial Sequence
  synthetic T1 epitope peptide
KQIINMWQEVGKSMYA

SEQ ID NO: 21 - protein, 16 aa, Artificial Sequence
  synthetic T1 epitope peptide
KQIINMWQEVGKAAYA

SEQ ID NO: 22 - protein, 16 aa, Artificial Sequence
  synthetic T1 epitope peptide
KQIINMWQEVGKAMAA

SEQ ID NO: 23 - protein, 16 aa, Artificial Sequence
  synthetic T1 epitope peptide
KQIINMWQEVGKAMFA

SEQ ID NO: 24 - protein, 16 aa, Artificial Sequence
  synthetic T1 epitope peptide
KQIINMWQEVGKAMIA

SEQ ID NO: 25 - protein, 16 aa, Artificial Sequence
  synthetic T1 epitope peptide
KQIINMWQEIGRAMAA

SEQ ID NO: 26 - protein, 24 aa, Artificial Sequence
  synthetic PCLUS 3 peptide
KQIINMWQEVGKAMYAPPISGQIR

SEQ ID NO: 27 - protein, 24 aa, Artificial Sequence
  synthetic PCLUS 3 peptide
KQIINMWQEIGKAMYAPPISGQIR

SEQ ID NO: 28 - protein, 24 aa, Artificial Sequence
  synthetic PCLUS 3 peptide
KQIINMWQEVGRAMYAPPISGQIR

SEQ ID NO: 29 - protein, 24 aa, Artificial Sequence
  synthetic PCLUS 3 peptide
KQIINMWQEVGKAMAAPPISGQIR

SEQ ID NO: 30 - protein, 24 aa, Artificial Sequence
  synthetic PCLUS 3 peptide
KQIINMWQEIGRAMAAPPISGQIR

SEQ ID NO: 31 - protein, 13 aa, Artificial Sequence
  synthetic p18 peptide from HIV-1 IIIB
RIQRGPGRAFVTI

SEQ ID NO: 32 - protein, 13 aa, Artificial Sequence
  synthetic p18 peptide from HIV-1 MN
RIHIGPGRAFYTT

SEQ ID NO: 33 - protein, 13 aa, Artificial Sequence
  synthetic p18 peptide from HIV-1 SC
SIHIGPGRAFYAT

SEQ ID NO: 34 - protein, 13 aa, Artificial Sequence
  synthetic p18 peptide from HIV-1 RF
SITKGPGRVIYAT

SEQ ID NO: 35 - protein, 13 aa, Artificial Sequence
  synthetic p18 peptide from HIV-1 SF2
SIYIGPGRAFHTT

SEQ ID NO: 36 - protein, 13 aa, Artificial Sequence
  synthetic p18 peptide from HIV-1 NY5
GIAIGPGRTLYAR

SEQ ID NO: 37 - protein, 13 aa, Artificial Sequence
  synthetic p18 peptide from HIV-1 CDC4
RVTLGPGRVWYTT

SEQ ID NO: 38 - protein, 13 aa, Artificial Sequence
  synthetic p18 peptide from HIV-1 WMJ2
SLSIGPGRAFRTR

SEQ ID NO: 39 - protein, 15 aa, Artificial Sequence
  synthetic p18 peptide from HIV-1 Z321
SISIGPGRAFFATTD

SEQ ID NO: 40 - protein, 15 aa, Artificial Sequence
  synthetic p18 peptide from HIV-1 Z3
SIRIGPGKVFTAKGG

SEQ ID NO: 41 - protein, 12 aa, Artificial Sequence
  synthetic p18 peptide from HIV-1 MAL
FGPGQALYTTGI

SEQ ID NO: 42 - protein, 15 aa, Artificial Sequence
  synthetic p18 peptide from HIV-1 Z6
STPIGLGQALYTTRG

SEQ ID NO: 43 - protein, 15 aa, Artificial Sequence
  synthetic p18 peptide from HIV-1 JYI
STPIGLGQALYTTRI

SEQ ID NO: 44 - protein, 15 aa, Artificial Sequence
  synthetic p18 peptide from HIV-1
RTPTGLGQSLYTTRS

SEQ ID NO: 45 - protein, 9 aa, Artificial Sequence
  synthetic HIV-1 reverse transcriptase epitope
VIYQYMDDL

SEQ ID NO: 46 - protein, 9 aa, Artificial Sequence
  synthetic IV9 epitope
ILKEPVHGV

SEQ ID NO: 47 - protein, 10 aa, Artificial Sequence
  synthetic SV10 epitope
SLLNATDIAV

SEQ ID NO: 48 - protein, 9 aa, Artificial Sequence
  synthetic SL9 epitope
SLYNTVATL

SEQ ID NO: 49 - protein, 9 aa, Artificial Sequence
  synthetic AL9 epitope
AFHHVAREL